(19) United States
(12) Patent Application Publication
(10) Pub. No.: US 2018/0056039 A1
(43) Pub. Date: Mar. 1, 2018

(54) METHOD FOR TRACKING AND POSITIONING MAGNETIC CATHETER AND STRUCTURE OF MAGNETIC CATHETER

(71) Applicant: NATIONAL CHENG KUNG UNIVERSITY, TAINAN CITY (TW)

(72) Inventors: CHING-HSING LUO, TAINAN CITY (TW); MENG-DAR SHIEH, TAINAN CITY (TW); CHIEN-CHEN CHANG, TAINAN CITY (TW); MING-HUNG TSAI, TAINAN CITY (TW); HSIN-EN FANG, TAINAN CITY (TW); YI ZHANG, TAINAN CITY (TW); YANG-BEN LIN, TAINAN CITY (TW); WEN-HORNG YANG, TAINAN CITY (TW); MING-CHANG SHIH, TAINAN CITY (TW); CHENG-CHI TAI, TAINAN CITY (TW)

(21) Appl. No.: 15/248,439

(22) Filed: Aug. 26, 2016

(51) Int. Cl. A61M 25/01; G01B 7/30

(52) U.S. Cl. CPC A61M 25/0127; A61M 2205/3317; G01B 7/30

(57) ABSTRACT

A method for tracking and positioning a magnetic catheter and a structure of a magnetic catheter are disclosed for facilitating tracking and positioning of catheters in human bodies without using electromagnetic induction as conventionally used. When electromagnetic induction and remote magnetic control are used together, their respective magnetic fields may mutually interfer, increasing the risk of operational errors of the magnetic catheters they are working on. The disclosed magnetic catheter has an elastic unit. While the magnetic catheter bends, inductance variation caused by elastic deformation of the elastic unit is measured for calculating an actual bending angle of the magnetic catheter. Then the motion of the magnetic catheter can be amended accordingly. By using calculation instead of electromagnetic tracking, mutual interference between different magnetic fields can be prevented.

### BACKGROUND OF THE INVENTION

### 1. Technical Field

The present invention relates to a method for tracking and positioning a magnetic catheter and a structure of a magnetic catheter, and more particularly to using calculation instead of electromagnetic tracking to determine the actual bending angle of a magnetically controlled catheter.

### 2. Description of Related Art

Conventionally for tracking and positioning a catheter in a patient's body, electromagnetic tracking is used to add a gradient magnetic field outside the patient's body, so that an inductive magnetic field produced by the interaction between the coil on the catheter and the gradient magnetic field, the coordinate position of the catheter in the patient's body can be determined. This technology has been reported by H. D. Becker in his paper “Electromagnetic navigation for peripheral lung lesions and mediastinal lymph nodes” Emmanuel Wilson has published “Accuracy Analysis of Electromagnetic Tracking within Medical Environments” in which the accuracy of electromagnetic tracking for madical use is discussed. Emmanuel Wilson, et al. in their work titled “A Buyer's Guide to Electromagnetic Tracking Systems for Clinical Applications” investigate the selection among existing electromagnetic tracking systems for clinical applications, and provide detailed description to the structure of a electromagnetic tracking system.

Magnetic control for catheters is also a popular field to develop. Currently, a magnetic member is attached to a catheter's front end so that the magnetic member and in turn the catheter can be controlled by varying the ambient magnetic field. This allows the catheter to bend and reach different sites in a human body. For example, U.S. Pat. No. 6,537,196 titled “MAGNET ASSEMBLY WITH VARIABLE FIELD DIRECTIONS AND METHODS OF MAGNETICALLY NAVIGATING MEDICAL OBJECTS” involves rotating plural magnets to change direction of the resultant magnetic field. U.S. Pat. No. 6,311,082 titled “DIGITAL MAGNETIC SYSTEM FOR MAGNETIC SURGERY” differently uses plural electromagnets and vary the magnetic field in terms of strength and direction by changing currents applied to the electromagnets.

However, when remote magnetic control (RMC) is used together with electromagnetic tracking for positioning the catheter, the two additional magnetic fields can interfere with each other, leading to operational errors.

### SUMMARY OF THE INVENTION

Hence, the objective of the present invention is to provide a method for tracking and positioning a magnetic catheter that eliminates the use of electromagnetic measurement for tracking and positioning the magnetic catheter, so as to prevent mutual interference between magnetic fields for electromagnetic measurement and for magnetic control.

The magnetic catheter has a front section being a flexible section, and the flexible section has a free end provided with a magnetic member. The method comprises the following steps:

A. applying a magnetic field to the magnetic member so as to make the flexible section of the magnetic catheter perform a bending motion, and measuring variation of an inductance value caused by elastic deformation of an elastic unit on the flexible section, wherein the elastic deformation is generated in response to the bending motion of the flexible section; B. inputting the variation of the inductance value into a processing unit so that the processing unit calculates an actual bending angle of the flexible section based on the variation of the inductance value; and C. comparing the actual bending angle to a preset bending angle, and adjusting the actual bending angle of the flexible section to make the actual bending angle become equal to the preset bending angle.

Further, the flexible section and a rear section of the magnetic catheter have different rigidities due to the fact that they are made of different materials. Alternatively, the flexible section is a multi joint section with each joint thereof having a single bending degree of freedom so that the multi joint section performs the bending motion in a direction of the bending degree of freedom. Furthermore, the multi joint section has a first side and a second side opposite to the first side, and the bending degree of freedom of the multi joint section allows the multi joint section to perform the bending motion toward the first side or the second side, so that in Step A, when the multi joint section of the catheter bends from the first side toward the second side, the variation of the inductance value caused by elongation of the elastic unit at the first side or/and the variation of the inductance value caused by contraction of the elastic unit at the second side are measured, and when the multi joint section of the catheter bends from the second side toward the first side, the variation of the inductance value caused by elongation of the elastic unit at the second side or/and the variation of the inductance value caused by contraction of the elastic unit at the first side are measured.

The present invention further provides a magnetic catheter for working with the method as described above. The magnetic catheter has a front end being a multi joint section with each joint thereof having a single bending degree of freedom, and the multi joint section has a free end provided with a magnetic member, so that by applying a magnetic field to the magnetic member, the multi joint section is controlled to perform a bending motion, wherein:

the multi joint section includes a plurality of joints that are pivotally connected one by one, and each of two adjacent said joints has an inclined abutting surface that faces the inclined abutting surface of the other, so that when the multi joint section performs the bending motion in the direction of the bending degree of freedom, the abutting surfaces of each two adjacent said joints abut on each other, in which the joint closer to the free end has the abutting surface inclined more, and an elastic unit is combined to the multi joint section so that in response to the bending motion of the multi joint section, the elastic unit performs elastic deformation.

Further, among the joints of the multi joint section, the one closer to the free end is shorter.

Further, the elastic unit is connected between two ends of the multi joint section. Furthermore, the multi joint section has a first side and a second side opposite to the first side, and the degree of freedom of the multi joint section allows the multi joint section to perform the bending motion toward the first side or the second side. Moreover, the elastic unit comprises a first elastic member combined to the first side of the multi joint section, and a second elastic member combined to the second side of the multi joint section.

Further, a sensing circuit is connected to the elastic unit for measuring variation of an inductance value caused by the elastic deformation of the elastic unit, and a processing unit electrically connected to the sensing circuit for receiving and using the variation of the inductance value to calculate an actual bending angle of the multi-joint section.

According to at least one of the features described above, the following effects can be achieved:

1. The actual bending angle of the magnetic catheter is learned from the variation of the inductance value caused by the elastic deformation of the elastic unit in response to the bending motion of the magnetic catheter, which is a closed-loop control, thereby eliminating the need of another magnetic field as otherwise generated by electromagnetic tracking in the prior art. This is perfect for tracking and positioning magnetically controlled catheters, and is free from the problem related to mutual interference between different magnetic fields.

2. As compared to the prior art that uses electromagnetic tracking to track and position a catheter in a human body, the present invention is more advantageous thanks to its simple configuration and high realizability.

3. From the given amounts of feed and rotation of the magnetic catheter and the actual bending angle of the magnetic catheter as calculated, the position of the magnetic catheter inside a patient's body can be precisely determined.

### BRIEF DESCRIPTION OF THE DRAWINGS

FIG. 1A is an applied view of a first type of magnetic control according to the present invention.

FIG. 1B is another applied view of the first type of magnetic control according to the present invention.

FIG. 2A is an applied view of a second type of magnetic control according to the present invention.

FIG. 2B is another applied view of the second type of magnetic control according to the present invention.

FIG. 2C is still another applied view of the second type of magnetic control according to the present invention.

FIG. 3A is an applied view of a third type of magnetic control according to the present invention.

FIG. 3B is another applied view of the third type of magnetic control according to the present invention.

FIG. 3C is still another applied view of the third type of magnetic control according to the present invention.

FIG. 4A is an applied view of a fourth type of magnetic control according to the present invention.

FIG. 4B is another applied view of the fourth type of magnetic control according to the present invention.

FIG. 4C is still another applied view of the fourth type of magnetic control according to the present invention.

FIG. 5A is an applied view of a fifth type of magnetic control according to the present invention.

FIG. 5B is another applied view of the fifth type of magnetic control according to the present invention.

FIG. 6 is a perspective view according to one embodiment of the present invention, showing a multi joint section of a magnetic catheter being disposed in a resultant magnetic field.

FIG. 7 is a side view showing the multi joint section of the magnetic catheter in FIG. 6.

FIG. 8 is a schematic drawing according to one embodiment of the present invention, showing that the multi joint section of the magnetic catheter performs a bending motion under the acting force produced from an annular region, in the resultant magnetic field, which has a relative high magnetic flux density, wherein the resultant magnetic field is generated between the like poles of the two magnets.

FIG. 9 is a schematic drawing according to one embodiment of the present invention, showing how the multi joint section bends in the case that the resultant magnetic field is not moved while the multi joint section of the magnetic catheter performs the bending motion.

FIG. 10 is a schematic drawing according to the embodiment in FIG. 9, representing the relationship between the current required and the bending angle in the case that the resultant magnetic field is not moved synchronously while the multi joint section of the magnetic catheter performs the bending motion.

FIG. 11 is a schematic drawing according to one embodiment of the present invention, showing how the multi joint section bends in the case that the magnetic member is retained within the magnetic annulus by moving the resultant magnetic field while the multi joint section of the magnetic catheter performs the bending motion.

FIG. 12 is a schematic drawing according to the embodiment in FIG. 11, representing the relationship between the current required and the bending angle in the case that the magnetic member is retained within the magnetic annulus by moving the resultant magnetic field while the multi joint section of the magnetic catheter performs the bending motion.

FIG. 13 is a schematic drawing according to one embodiment of the present invention, representing the relationship between the currents for the electromagnets and the bending angle of the magnetic catheter, in which the strength of the resultant magnetic field is gradually reduced until the direction of the resultant magnetic field is changed so as to return the multi joint section of the magnetic catheter.

FIG. 14 is a schematic view according to the embodiment in FIG. 13, showing how the multi joint section of the magnetic catheter is returned to its initial state while being pulled by the resultant magnetic field whose direction has been changed.

FIG. 15 is a perspective view of the magnetic catheter according to one embodiment of the present invention, depicting an exemplificative structure of the magnetic catheter for practical use.

FIG. 16 is another perspective view of the magnetic catheter of FIG. 15, showing how the magnetic catheter performs a bending motion.

FIG. 17 is a schematic view according to one embodiment of the present invention, showing how the elastic elements combined with the multi joint section of the magnetic catheter detect the actual bending angle.

FIG. 18 is a flow chart according to one embodiment of the present invention, explaining how the elastic elements combined with the multi joint section of the magnetic catheter detect the actual bending angle that is to be compared with the preset bending angle.

FIG. 19 is a schematic drawing illustrating the multi joint section of the magnetic catheter bending from the first side toward the second side.

FIG. 20 is a schematic drawing illustrating the multi joint section of the magnetic catheter bending from the second side toward the first side.

### DETAILED DESCRIPTION OF THE INVENTION

For further illustrating the means and functions on which the present invention achieves the certain objectives, the following description, in conjunction with the accompanying drawings and preferred embodiments, is set forth as below to illustrate the implement, structure, features and effects of the subject matter of the present invention.

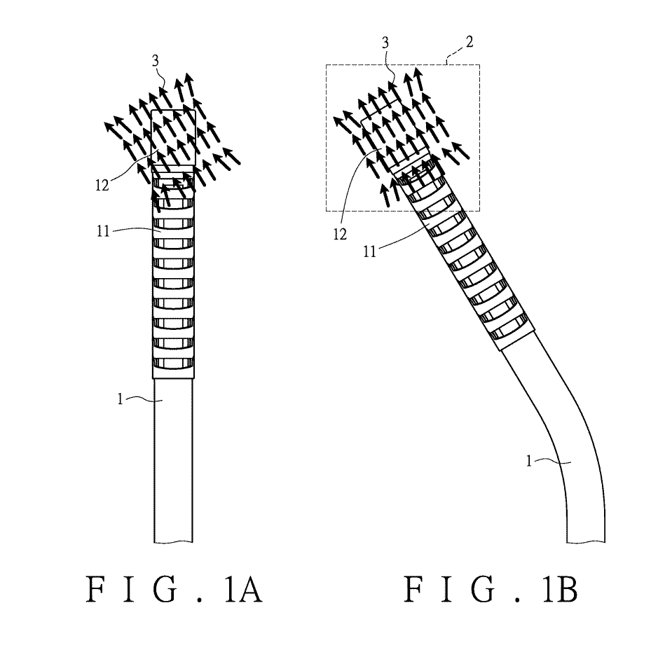

Referring to FIG. 1A, in the present embodiment, a magnetic catheter (1) has a front section formed as a flexible section. According to the present embodiment, the flexible section is a multi joint section (11) with each joint thereof having a single bending degree of freedom. At a free end of the multi joint section (11), a magnetic member (12) is provided. The magnetic member (12) is an axial magnet. Therein, the magnetic catheter (1) is capable of performing a feeding motion and a rotating motion along a linear first route. The first route is an extending route of the magnetic catheter (1).

In embodiments of the present invention, five types of magnetic control are applicable.

The first type is as shown in FIG. 1A and FIG. 1B.

In Step A, a target site (2) is set, as shown in FIG. 1B.

In Step B, at least two magnets are set opposite and separated from each other by a proper distance so as to form a resultant magnetic field (3). The resultant magnetic field (3) is applied to the multi joint section (11) of the magnetic catheter (1) and has a direction pointing toward the target site (2), while at this time the target site (2) is in a direction different from the direction of the bending degree of freedom.

In Step C, the magnetic catheter (1) is controlled not to perform the feeding motion and the rotating motion, and the magnetic member (12) is thus driven by the resultant magnetic field (3) to make the magnetic catheter (1) perform a declination, thereby making the free end of the multi joint section (11) point toward the target site (2).

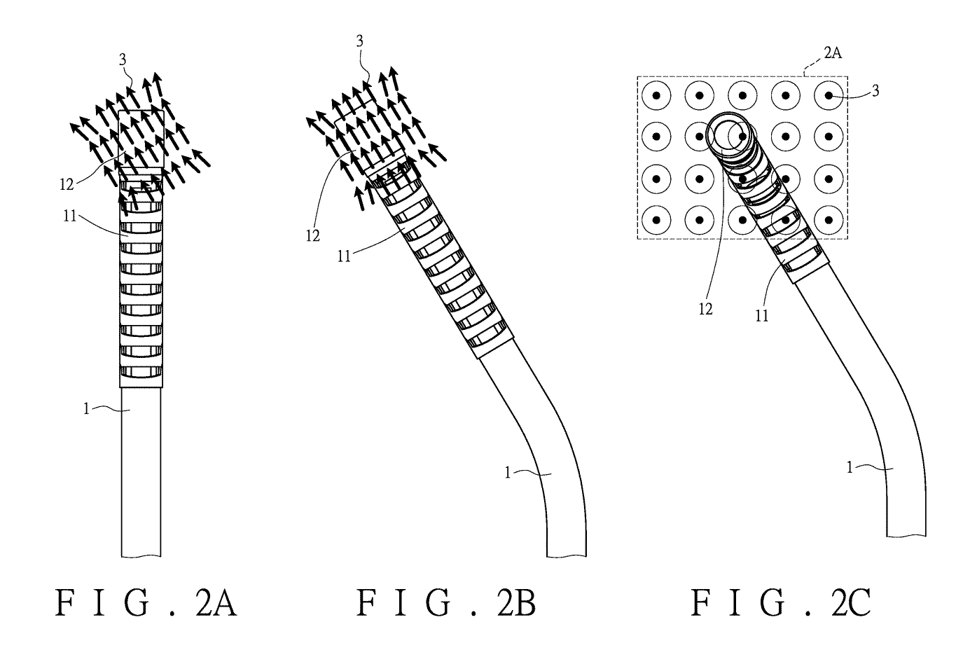

The second type is as illustrated in FIG. 2A through FIG. 2C.

This type has an addition step after the declination of the magnetic catheter (1) as described in the first type. The addition step, step D, involves changing the direction of the resultant magnetic field (3) again to make the resultant magnetic field (3) point toward the bending degree of freedom, so that the magnetic member (12) can be driven by the resultant magnetic field (3) to lead the multi joint section (11) to perform a bending motion along the bending degree of freedom. The multi joint section (11) can thereby be in a three-dimensional torsion state, as shown in FIG. 2C, with the free end thereof pointing toward another target site (2A).

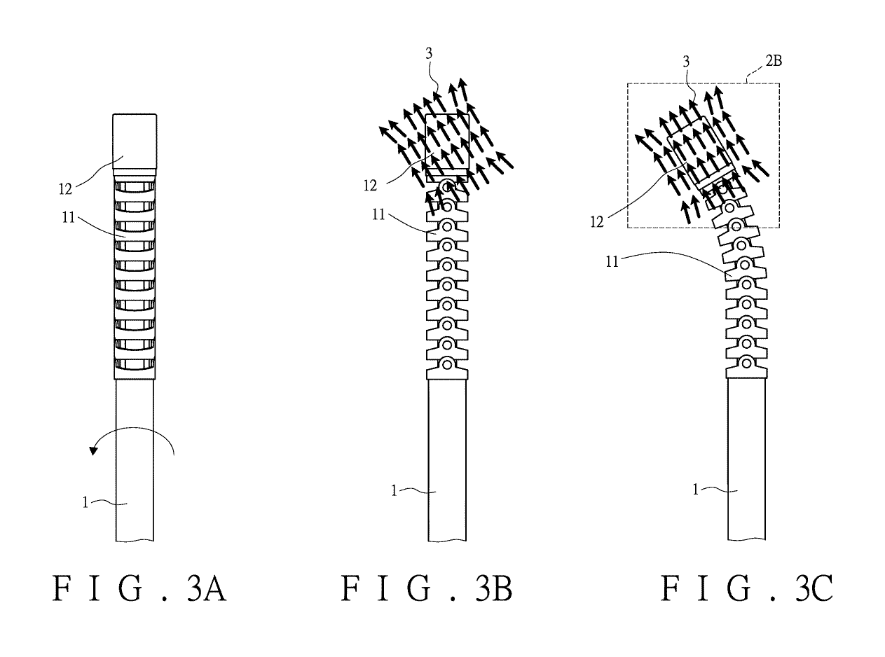

The third type is as illustrated in FIG. 3A through FIG. 3C.

In Step A, the magnetic catheter (1) is such rotated that a target site (2B) is set in the direction of the bending degree of freedom of the multi joint section (11).

In Step B, the resultant magnetic field (3) is applied to the multi joint section (11) and has its direction pointing toward the target site (2B).

In Step C, the magnetic catheter (1) is controlled not to perform the feeding motion and the rotating motion, and the magnetic member (12) is thus driven by the resultant magnetic field (3) to make the multi joint section (11) of the endoscopic catheter (1) perform a bending motion along the bending degree of freedom, thereby making the free end point toward the target site (2B).

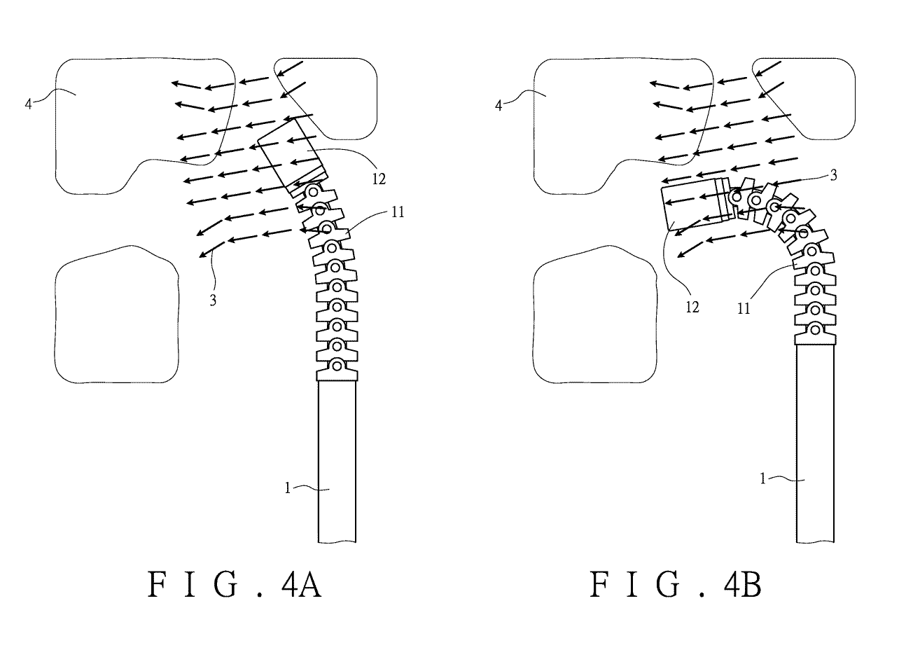

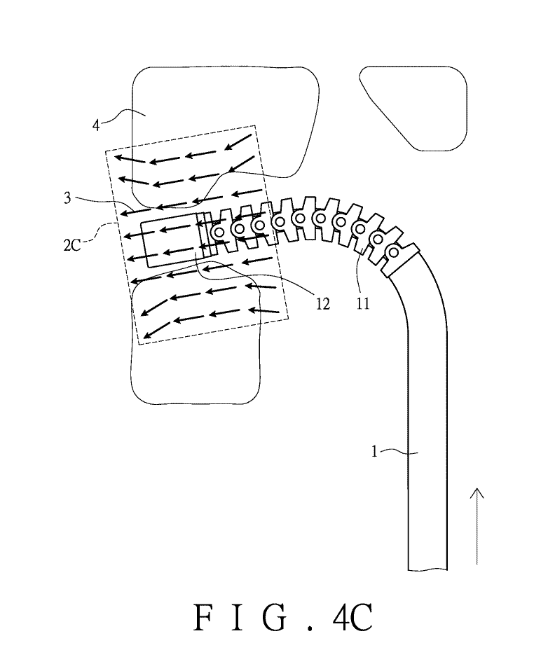

The fourth type is as illustrated in FIG. 4A through FIG. 4C.

In Step A, the magnetic catheter (1) enters a body cavity (4), and a target site (2C) is set. The target site (2C) is located in the direction of the bending degree of freedom of the multi joint section (11).

In Step B, the resultant magnetic field (3) is applied to the multi joint section (11) of the magnetic catheter (1) and has a direction pointing toward the target site (2C).

In Step C, the magnetic catheter (1) is controlled not to perform the feeding motion and the rotating motion, and the multi joint section (11) thus performs a bending motion to avoid obstacles.

In Step D, the resultant magnetic field (3) is moved while the magnetic catheter (1) is controlled to perform the feeding motion, so that the magnetic member (12) is driven by the resultant magnetic field (3) to control the free end of the multi joint section (11) to reach the target site (2C).

In addition to the method described above, by shifting the resultant magnetic field (3) and controlling the magnetic catheter (1) to perform the feeding motion, the free end of the multi joint section (11) can linearly advance toward and reach a desired target site.

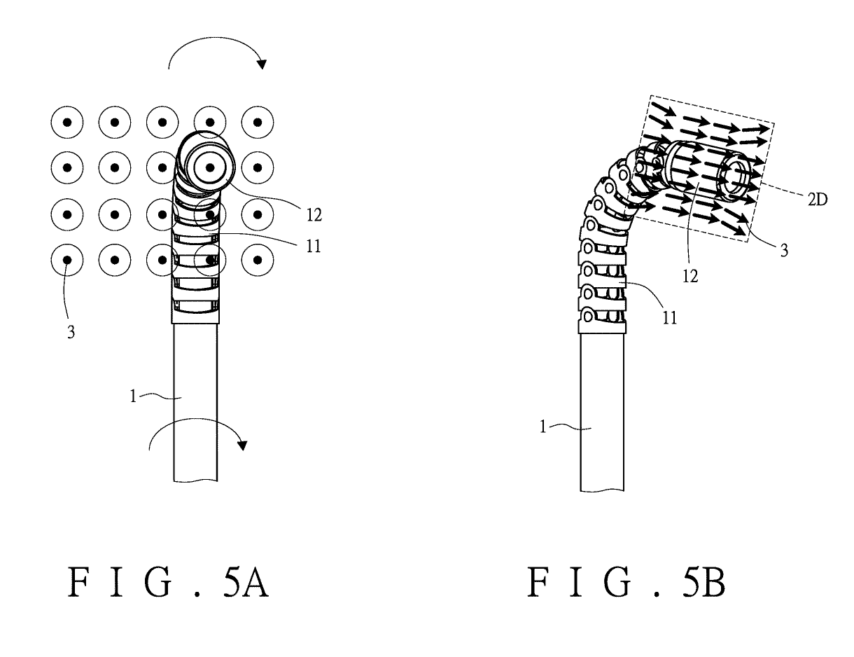

The fifth type is as illustrated in FIG. 5A and FIG. 5B.

In Step A, a target site (2D) is set.

In Step B, the resultant magnetic field (3) is applied to the multi joint section (11) of the magnetic catheter (1) and has a direction pointing toward the direction of the bending degree of freedom.

In Step C, the magnetic catheter (1) is controlled not to perform the feeding motion and the rotating motion, and the multi joint section (11) of the magnetic catheter (1) thus performs a bending motion in the direction of the bending degree of freedom.

In Step D, the resultant magnetic field (3) is rotated, and the magnetic catheter (1) is also rotated according to the direction of the resultant magnetic field (3), so that the direction of the resultant magnetic field (3) is aligned with the direction of the bending degree of freedom of the multi joint section (11), thereby driving the free end of the multi joint section (11) to point toward the target site (2D).

The application of the resultant magnetic field (3) and the synchronous control of the feeding and rotating motions of the magnetic catheter (1) jointly ensure that the free end of the multi joint section (11) can selectively point toward any one of the target sites (2)(2A)(2B)(2C)(2D). With the cooperation of the feeding and rotating motions of the magnetic catheter (1), the magnetic member (12) is prevented from becoming uncontrollable to the resultant magnetic field (3), which may otherwise causes unexpected operational errors when the resultant magnetic field (3) shifts or changes direction. More specifically, without synchronously feeding or rotating the magnetic catheter (1) according to the movement or the direction of the resultant magnetic field (3), the magnetic catheter (1) could be twisted and thus generate considerable resistance that hinders the multi joint section (11) from following the resultant magnetic field (3). The free end of the multi joint section (11) then could fail to reach the target site and even come out of the control of the resultant magnetic field (3). While the present invention is effective in overcoming this problem, the solution is not limited to that described above and can be designed by varying the resultant magnetic field (3) and feeding/rotating the magnetic catheter (1) according to any desired target site. The present invention thus provides interventional or endoscopic surgery with a method of reaching nidi quickly and precisely through magnetic control.

It is to be noted that instead of making the flexible section as the multi joint section (11), the present invention may have the flexible section and the rear section of the magnetic catheter (1) made of materials of different rigidities.

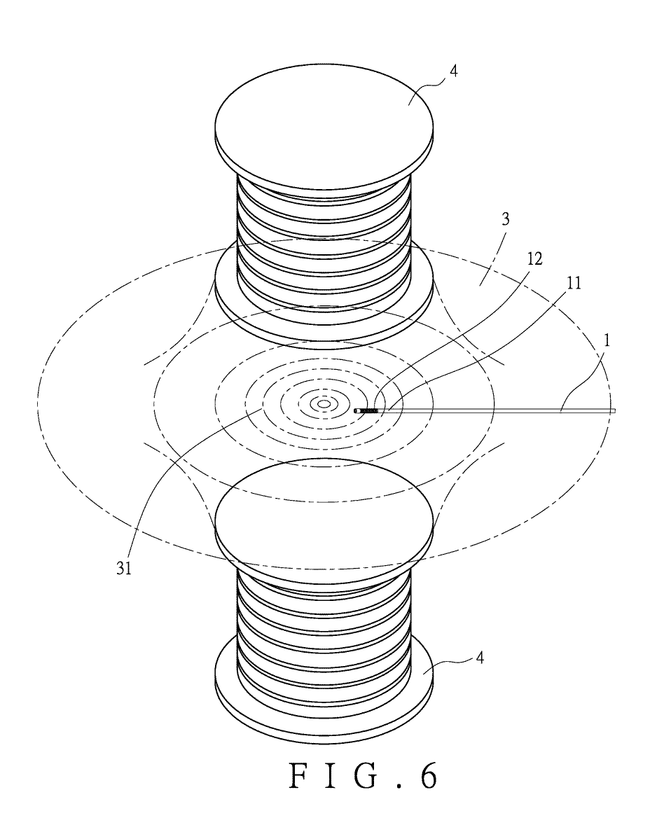

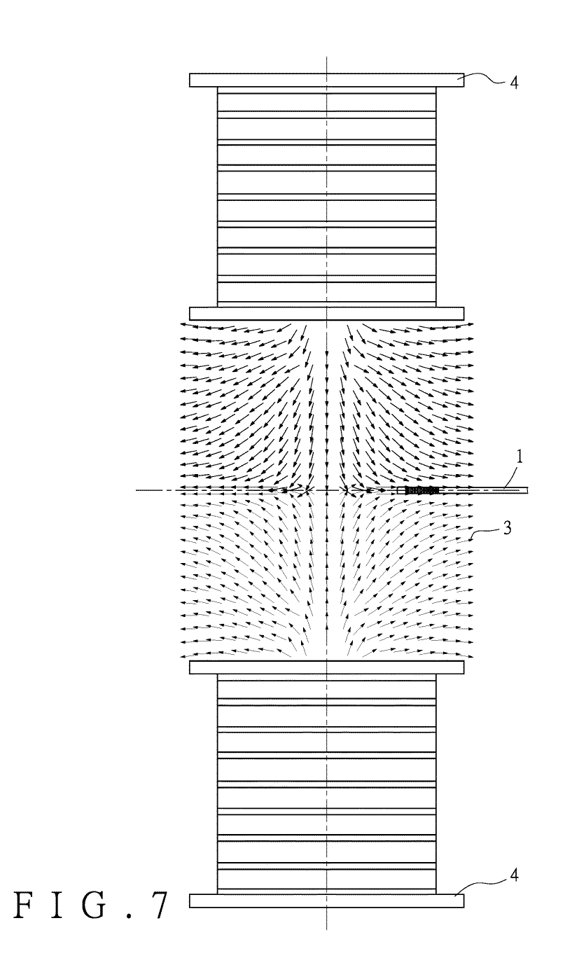

Referring to FIG. 6 through FIG. 8, the resultant magnetic field (3) is generated between two magnets, and an annular region therein is defined as a magnetic annulus (31). Generally, the magnetic annulus (31) refers to the region that has highest magnetic flux density on an acting plane, for example the region involving top 50 percent of the highest magnetic flux densities. When the multi joint section (11) of the magnetic catheter (1) enters the magnetic annulus (31) of the resultant magnetic field (3), a magnetic force is produced by the interaction between the magnetic member (12) and the magnetic annulus (31). Therein, the magnetic annulus (31) of the resultant magnetic field (3) is generated by the two magnets whose like poles face each other. For convenient control over the strength and direction of the resultant magnetic field (3), the magnets may be electromagnets (5).

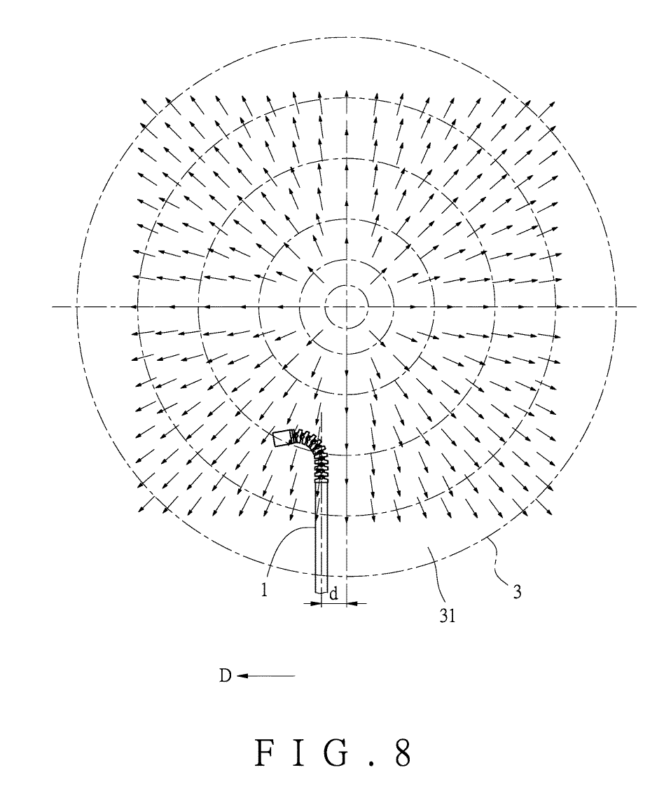

Referring to FIG. 8, the direction of the bending degree of freedom of the multi joint section (11) is pointed toward a desired direction (D). At this time, a magnetic force is produced by the interaction between the magnetic member and the magnetic annulus (31). Since the magnetic annulus (31) of the resultant magnetic field (3) is generated by the like poles of the two magnets (5), a head portion of the magnetic member (12), when entering the magnetic annulus (31), is repelled due to repulsion between the two like poles and in turn drives the multi joint section (11) to bend in the direction of the bending degree of freedom, making the free end of the multi joint section (11) advance in the desired direction (D).

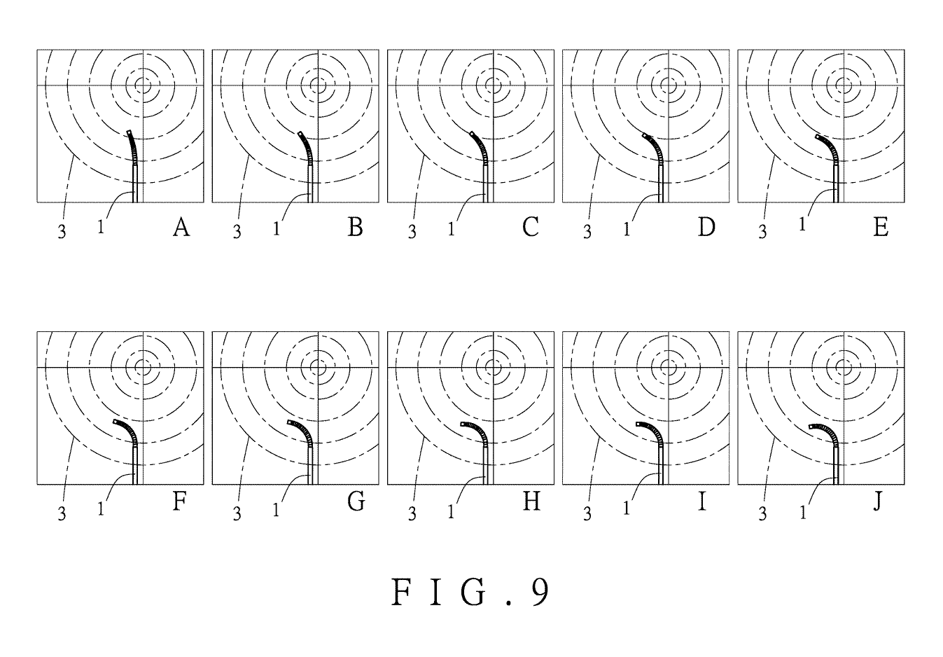

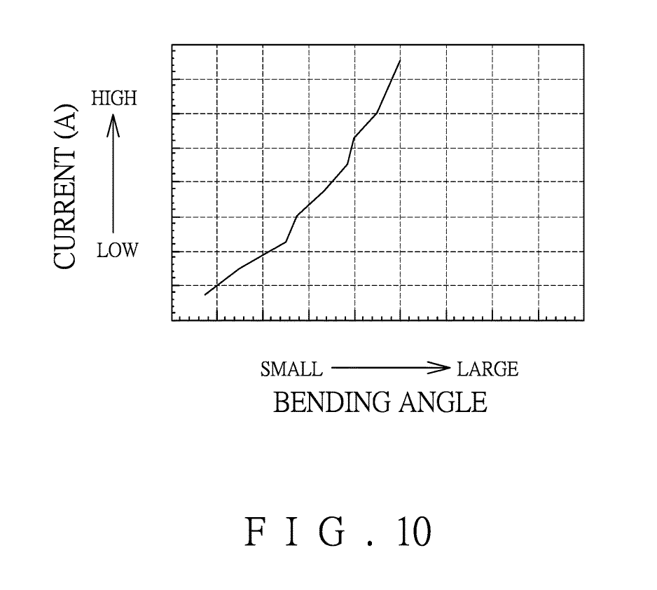

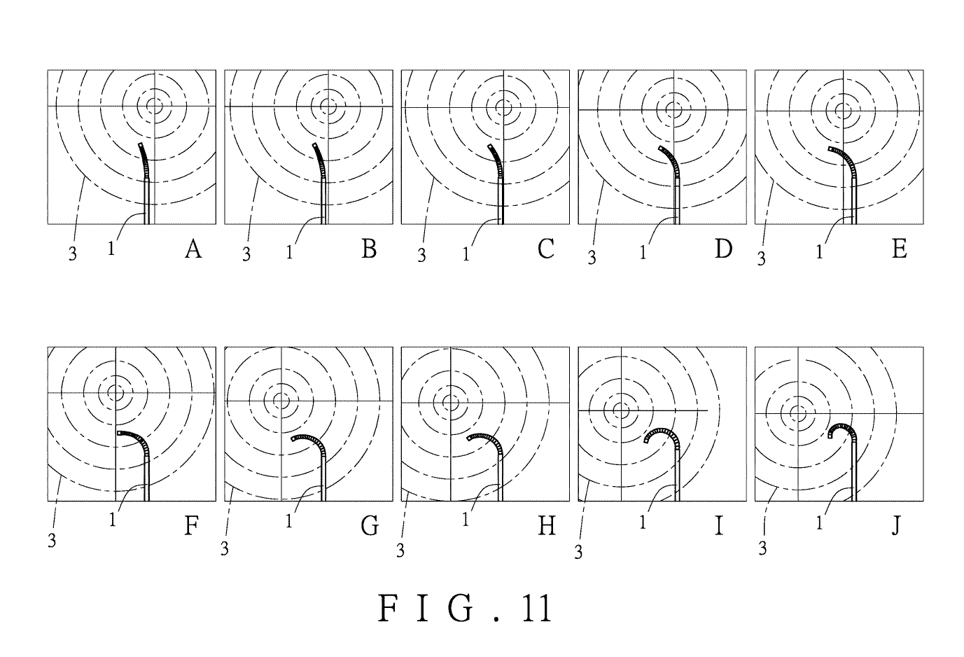

Referring to FIG. 9 and FIG. 10, thanks to its structure or/and material, the multi joint section (11) is resilient and, therefore, can generate a resilient returning force against the magnetic force. Therefore, when the multi joint section (11) performs the bending motion, the currents for the electromagnets (5) have to be gradually increased, so as to make the strength of the resultant magnetic field (3) gradually increase, thereby increasing the angle on which the multi joint section (11) bends. Further, in the case that the two opposite electromagnets (5) are not moved synchronously when the multi joint section (11) performs the bending motion, higher currents for the electromagnets (5) might be required in order to enhance the magnetic force because the magnetic member (12) of the multi joint section (11) could enter a region having lower magnetic flux density than the magnetic annulus (31).

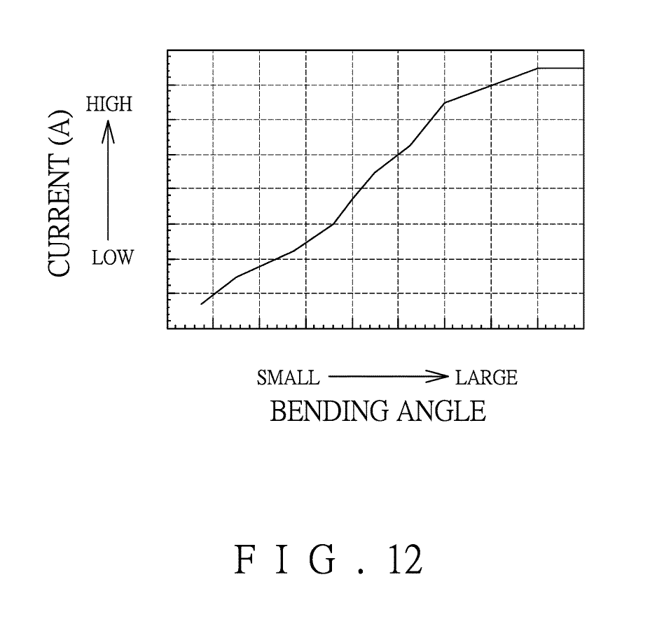

Referring to FIG. 11 and FIG. 12, in the event that the magnetic member (12) of the multi joint section (11) is retained in the magnetic annulus by moving the two electromagnets (5) while the multi joint section (11) performs the bending motion, the multi joint section (11) can perform the bending motion easier and even bend by a larger angle, thanks to the repulsion between the like poles of the two electromagnets (5). Moreover, the multi joint section (11) is bendable in the direction perpendicular to an extending direction of the two electromagnets (5) and is allowed to bend in multiple directions in the case that each joint thereof has a respective bending degree of freedom which is different from another joint thereof.

Referring to FIG. 10 and FIG. 12, when the currents for the two electromagnets (5) are fixed, a wider bending angle can be achieved in the case that the two electromagnets (5) are moved synchronously with the multi joint section (11), in comparison to the case that the two electromagnets (5) are not moved synchronously.

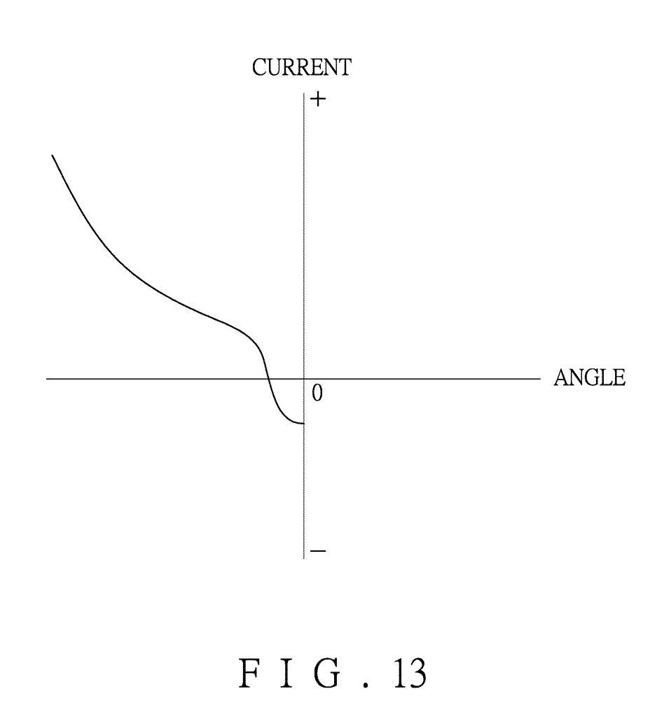

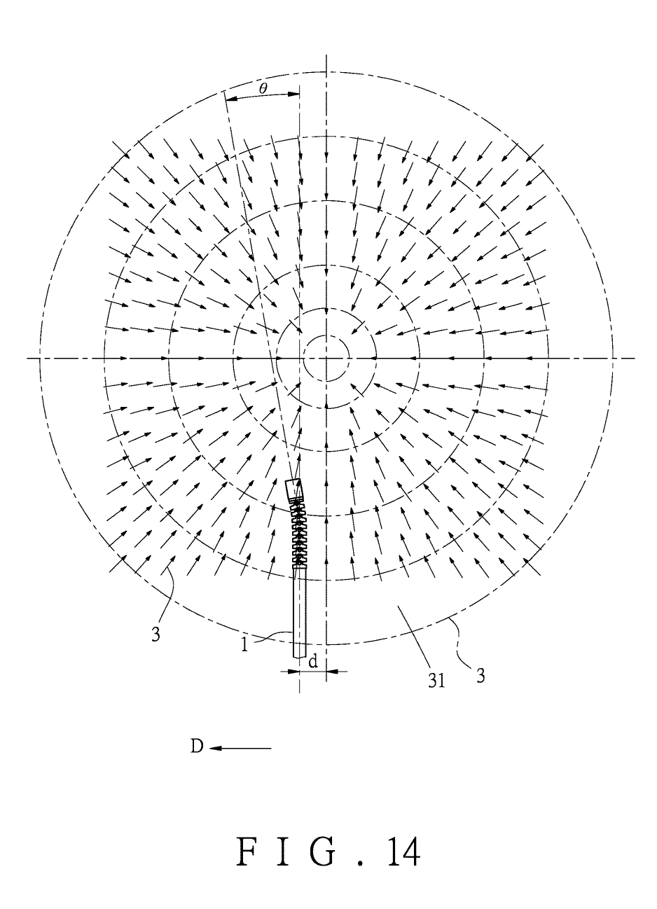

Referring to FIG. 13 and FIG. 14, for making the multi joint section (11) return to its initial state, the currents for the electromagnets (5) are gradually reduced, so as to make the strength of the resultant magnetic field (3) gradually decrease, thereby lowering the thrust acting on the multi joint section (11) and allowing the multi joint section (11) to gradually return due to its own resilient returning force. As a result of a natural physical phenomenon, the multi joint section (11) is not directly returned to its initial extending direction. Thus, after the multi joint section (11) has returned to a preset angle (0), such as an angle between 10 and 30 degrees, the resultant magnetic field (3) is reversed so as to change the direction of the magnetic force and generate a pull force applied on the multi joint section (11), thereby making the multi joint section (11) return to its initial state exactly.

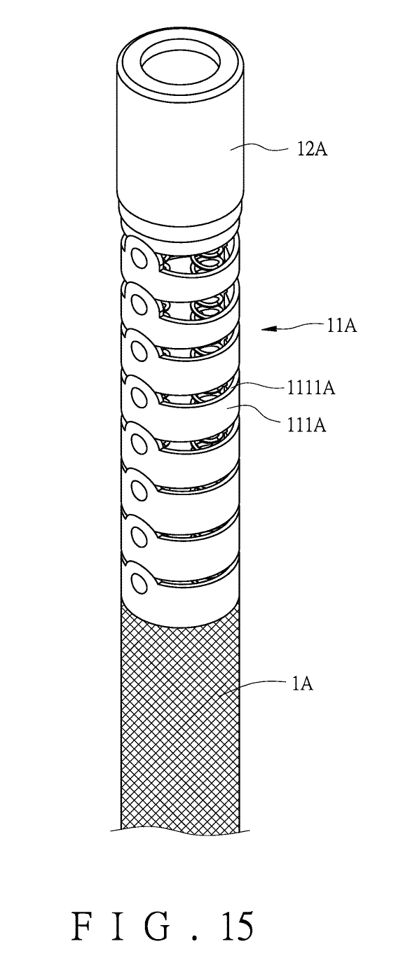

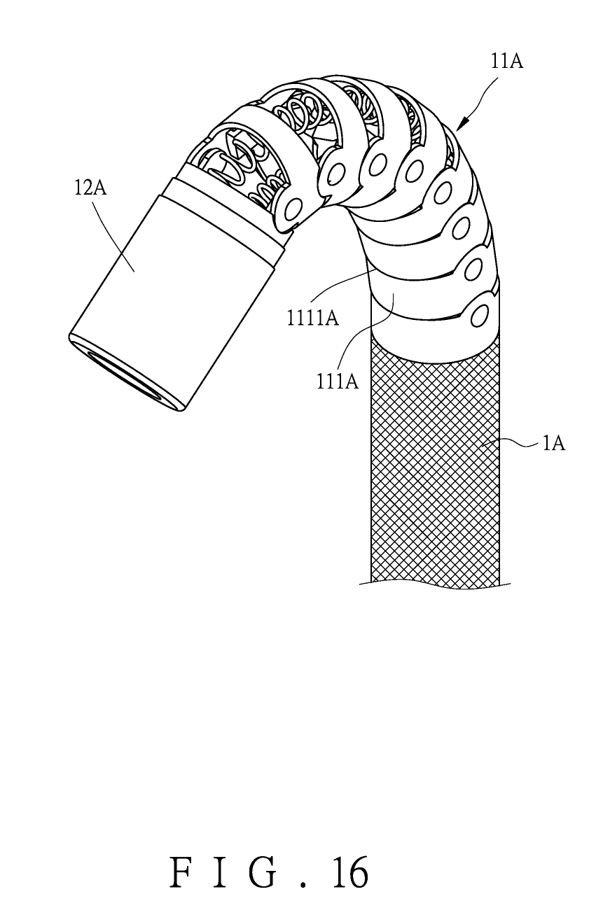

Referring to FIG. 15 and FIG. 16, for making the bending motion of the disclosed magnetic catheter meets the requirement of clinical use, an exemplificative structure of an endoscopic catheter (1A) is proposed.

The magnetic catheter (1A) has a front end provided with a multi joint section (11A) with each joint (111A) thereof having a single bending degree of freedom. At a free end of the multi joint section (11A), there is a magnetic member (12A). The joints (111A) of the multi joint section (11A) are pivotally connected one by one. Each of two adjacent said joints (111A) has an inclined abutting surface (1111A) that faces the inclined abutting surface (1111A) of the other, so that when the multi joint section (11A) performs the bending motion in the direction of the bending degree of freedom, the abutting surfaces (1111A) of each two adjacent said joints (111A) abut on each other. Preferably, the joint (111A) closer to the free end has its abutting surface (1111A) inclined more. In addition, among the joints (111A) of the multi joint section (11A), the one closer to the free end is shorter.

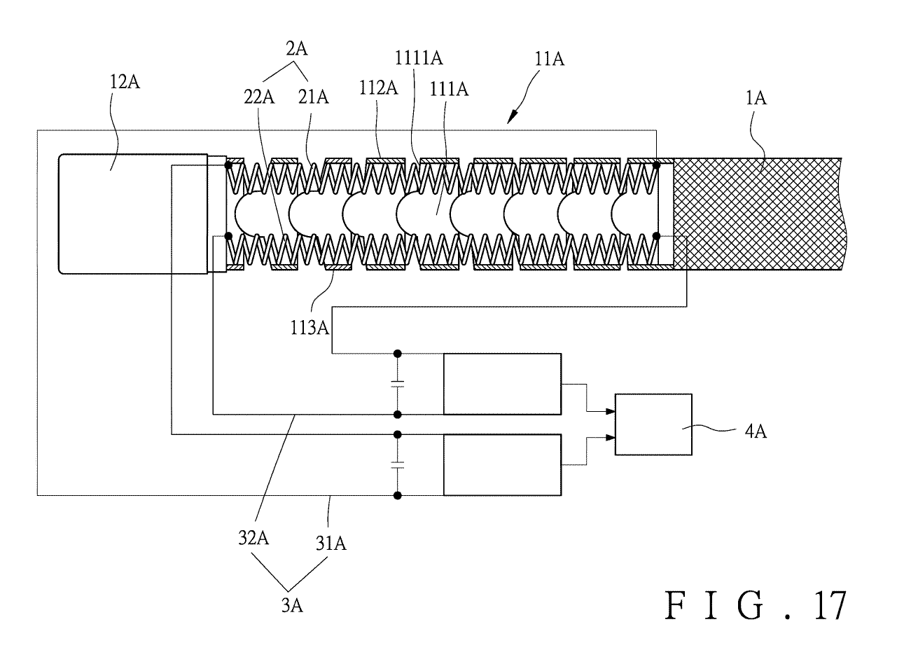

Referring to FIG. 17, furthermore, an elastic unit (2A) is provided between the two ends of the multi joint section (11A). When the multi joint section (11A) performs the bending motion, the elastic unit (2A) performs elastic deformation accordingly.

Referring to FIG. 17, the multi joint section (11A) has a first side (112A) and a second side (113A) opposite to the first side (112A). The bending degree of freedom of the multi joint section (11A) allows the multi joint section (11A) to bend toward either the first side (112A) or the second side (113A). In the present embodiment, the elastic unit (2A) comprises a first elastic member (21A) combined with the first side (112A) and a second elastic member (22A) combined with the second side (113A). There are also a sensing circuit (3A) and a processing unit (4A) that is electrically connected to the sensing circuit (3A). The sensing circuit (3A) comprises a first sensing circuit (31A) connected to the first elastic member (21A), and a second sensing circuit (32A) connected to the second elastic member (22A).

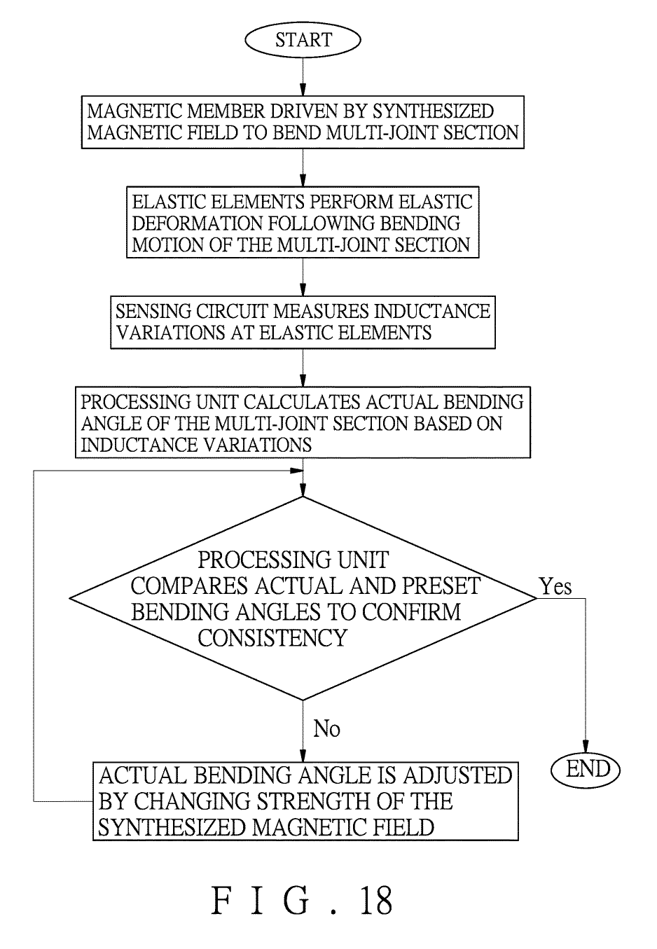

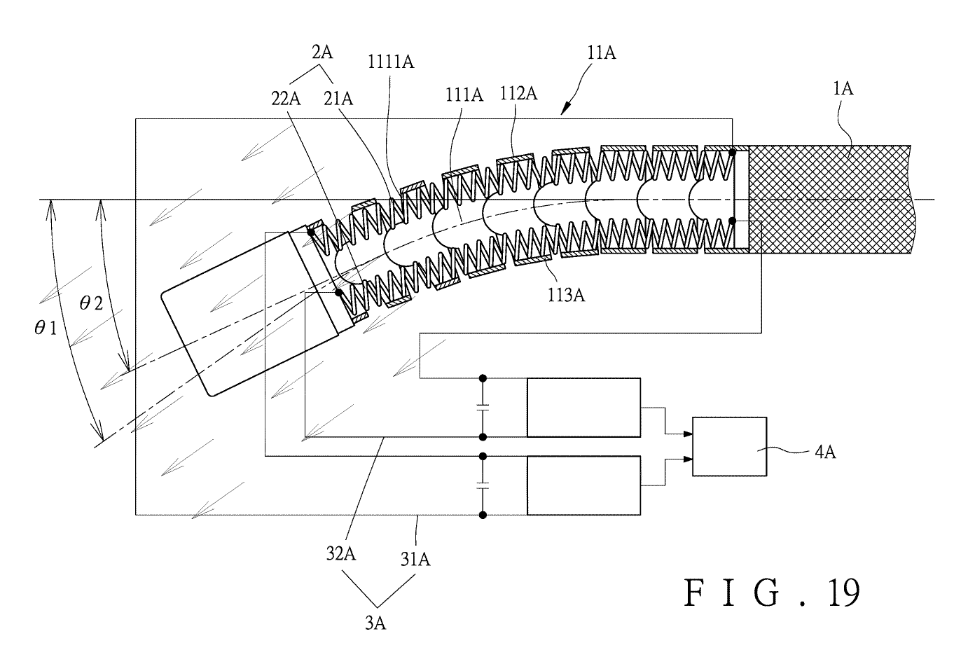

Now the reference is made to FIG. 18 and FIG. 19.

In Step A, the strength of the resultant magnetic field (3) is set according to a preset bending angle (01), and the magnetic member (12A) is thus driven by the resultant magnetic field (3) to make the multi joint section (11A) perform the bending motion. As used herein, the preset bending angle (01) refers to an angle on which the multi joint section (11A) bends in a patient's body as expected by doctors, i.e., in a direction aligning with nidi. By reaching the preset bending angle (01), the multi joint section (11A) can make a certain target site visible and accessible to the doctors. When the resultant magnetic field (3) makes the multi joint section (11A) bend from the first side (112A) toward the second side (113A), the first elastic member (21A) performs elastic deformation and elongates, while the second elastic member (22A) performs elastic deformation and contracts. The first sensing circuit (31A) measures variation of the inductance value caused by the elongation of the first elastic member (21A) at the first side (112A), and the second sensing circuit (32A) measures variation of the inductance value caused by the contraction of the second elastic member (22A) at the second side (113A).

In Step B, the variation of the inductance value are input to the processing unit (4A), and the processing unit (4A) uses this information to calculate an actual bending angle (02) of the multi joint section (11A). Since the variations of the inductance values include the variation of the inductance value caused by the elongation of the first elastic member (21A), and the variation of the inductance value caused by the contraction of the second elastic member (22A), the processing unit (4A) can also use this information to determine whether the multi joint section (11A) correctly bends from the first side (112A) toward the second side (113A).

In Step C, the actual bending angle (02) and the preset bending angle (01) are compared so the doctors can determine whether the actual bending angle (02) coincides with the preset bending angle (01). If there is any inconsistency therebetween, this informational also enables the doctors to adjust the actual bending angle (02) of the multi joint section (11A) until the actual bending angle (02) becomes equal to the preset bending angle (01) which means the multi joint section (11A) advances toward the direction aligning with nidi.

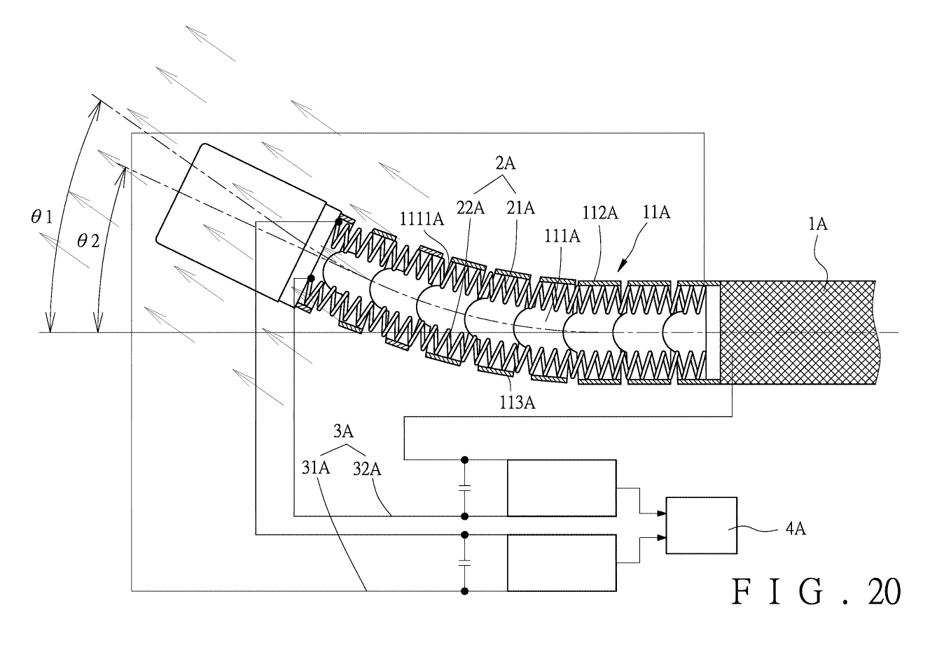

As shown in FIG. 20, when the multi joint section (11A) bends from the second side (113A) toward the first side (112A), the first elastic member (21A) is contracted due to elastic deformation while the second elastic member (22A) is elongated due to elastic deformation. The first sensing circuit (31A) measures variation of the inductance value caused by the contraction of the first elastic member (21A) at the first side (112A), and the second sensing circuit (32A) measures variation of the inductance value caused by the elongation of the second elastic member (22A) at the second side (113A). The subsequent comparison, determination and adjustment are similar to the previous embodiments and are not discussed in any length herein.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A method for tracking and positioning a magnetic catheter, the magnetic catheter having a front section provided with a flexible section, the flexible section having a free end provided with a magnetic member, and the method comprising the following steps:
A. applying a magnetic field to the magnetic member so as to make the flexible section of the magnetic catheter perform a bending motion, and measuring variation of an inductance value caused by elastic deformation of an elastic unit on the flexible section, wherein the elastic deformation is generated in response to a bending motion of the flexible section;
B. inputting the variation of the inductance value into a processing unit so that the processing unit calculates an actual bending angle of the flexible section based on the variation of the inductance value; and
C. comparing the actual bending angle to a preset bending angle, and adjusting the actual bending angle of the flexible section to make the actual bending angle become equal to the preset bending angle.

2. The method of claim 1, wherein the flexible section and a rear section of the magnetic catheter have different rigidities due to the fact that they are made of different materials.

3. The method of claim 1, wherein the flexible section is a multi joint section with each joint thereof having a single bending degree of freedom so that the multi joint section performs the bending motion in a direction of the bending degree of freedom.

4. The method of claim 3, wherein the multi joint section has a first side and a second side opposite to the first side, and the bending degree of freedom of the multi joint section allows the multi joint section to perform the bending motion toward the first side or the second side, so that in Step A, when the multi joint section of the catheter bends from the first side toward the second side, the variation of the inductance value caused by elongation of the elastic unit at the first side or/and the variation of the inductance value caused by contraction of the elastic unit at the second side are measured, and when the multi joint section of the catheter bends from the second side toward the first side, the variation of the inductance value caused by elongation of the elastic unit at the second side or/and the variation of the inductance value caused by contraction of the elastic unit at the first side are measured.

5. A magnetic catheter for working with the method of claim 1, wherein the flexible section is provided on a front end of the magnetic catheter and is a multi joint section with each joint thereof having a single bending degree of freedom so that the multi joint section is bendable when a magnetic field is applied to the magnetic member on the free end of the multi joint section; wherein the joints of the multi joint section includes are pivotally connected one by one, and each of two adjacent joints has an inclined abutting surface that faces the inclined abutting surface of the other, so that the abutting surfaces of each two adjacent joints abut on each other when the multi joint section performs the bending motion in the direction of the bending degree of freedom, in which the joint closer to the free end has the abutting surface inclined more, and an elastic unit is combined to the multi joint section so that the elastic unit performs elastic deformation in response to the bending motion of the multi-joint section.

6. The magnetic catheter of claim 5, wherein among the joints of the multi joint section, the one closer to the free end is shorter.

7. The magnetic catheter of claim 5, wherein the elastic unit is connected between two ends of the multi joint section.

8. The magnetic catheter of claim 7, wherein the multi joint section has a first side and a second side opposite to the first side, and the bending degree of freedom of the multi joint section allows the multi joint section to perform the bending motion toward the first side or the second side.

9. The magnetic catheter of claim 8, wherein the elastic unit comprises a first elastic member combined to the first side of the multi joint section and a second elastic member combined to the second side of the multi joint section.

10. The magnetic catheter of claim 5, further comprising a sensing circuit connected to the elastic unit for measuring variation of the inductance value caused by the elastic deformation of the elastic unit, and a processing unit connected to the sensing circuit for receiving and using the variation of the inductance value to calculate the actual bending angle of the multi joint section.
